(19) United States
(12) Patent Application Publication
(10) Pub. No.: US 2025/0248310 A1
(43) Pub. Date: Jul. 31, 2025

(54) SYSTEMS AND METHODS FOR INTEGRATING ULTRASONIC TRANSDUCERS WITH HYBRID CONTACTS

(71) Applicant: Exo Imaging, Inc., Santa Clara, CA (US)

(72) Inventors: Haesung KWON, Austin, TX (US); Brian BIRCUMSHAW, Oakland, CA (US); Sandeep AKKARAJU, Wellesley, MA (US)

(21) Appl. No.: 19/071,661

(22) Filed: Mar. 5, 2025

Related U.S. Application Data
(63) Continuation of application No. 17/159,538, filed on Jan. 27, 2021, now Pat. No. 12,274,174.
Application No. 17/159,538 is a continuation of application No. PCT/US2019/044528, filed on Jul. 31, 2019.
(60) Provisional application No. 62/713,272, filed on Aug. 1, 2018.

(51) Int. Cl. H10N 30/87; B06B 1/02; B06B 1/06; H10N 30/06; H10N 30/80

(52) U.S. Cl. CPC H10N 30/875; B06B 1/0207; B06B 1/0622; B06B 1/0666; H10N 30/06; H10N 30/802

(57) ABSTRACT

Disclosed herein are ultrasonic transducer systems with hybrid contacts comprising: an ultrasonic transducer element comprising a substrate and a membrane; an electrical circuitry; and one or more contacts connected to the ultrasonic transducer element and the electrical circuitry, wherein the one or more contacts are: designed geometrically using a set of rules; arranged with respect to the membrane based on the set of rules or a second set of rules, or both.

### CROSS-REFERENCE TO RELATED APPLICATIONS

This application is a continuation of U.S. patent application Ser. No. 17/159,538, filed Jan. 27, 2021, which is a continuation of International Patent Application No. PCT/US2019/044528, filed Jul. 31, 2019, which claims the benefit of U.S. Provisional Application No. 62/713,272, filed Aug. 1, 2018, which is hereby incorporated by reference in its entirety.

### BACKGROUND

An ultrasound transducer commonly includes a diaphragm, a substrate which forms a backing of the diaphragm, and contact(s) that connects the diaphragm to enable signal communication to and from the transducer.

Micromachined ultrasonic transducer (MUT) array(s) offer immense opportunity in the field of ultrasonics due to their efficiency in transducing between the electrical and acoustic energy domains.

### SUMMARY

A MUT chip integrated with a circuit, e.g., Application-specific integrated circuit (ASIC), features electrical contact(s) that is configured to transmit and receive signals to and from the MUT and ASIC. In addition, the electrical contact(s) can play a secondary but significant role in dictating the MUT dynamics because they act as a mechanical spring that impacts critical boundary conditions (e.g., mechanical boundary conditions such as how the MUT is attached to the MUT array, how the MUT array is anchored to the ASIC, and how the MUT is connected to the transmission media) of the MUT. As such, an integrated MUT with ASIC using only electrical contacts may destabilize the MUT and reduce dynamic performance of the MUTs.

The present disclosure includes systems and methods that enable MUT integration to an ASIC via hybrid contacts. A hybrid contact herein can enable both electrical connection as well as non-electrical connection, e.g., mechanical connection for the purpose of enhancing MUT dynamics, for example, magnitude of pressure output, surface velocity, and ultrasonic frequency bandwidth of the MUTs. The systems and methods herein can significantly enhance dynamic performance of MUTs. Such systems and methods may utilize one or more of: (1) adding additional mechanical contacts to the MUT, (2) arranging the contacts (electrical and/or mechanical), and (3) modifying the dimensions and shape of the contacts (electrical and/or mechanical).

In one aspect, disclosed herein are ultrasonic transducer systems with hybrid contacts comprising: an ultrasonic transducer element comprising a substrate and a membrane; an electrical circuitry; and one or more contacts connected to the ultrasonic transducer element and the electrical circuitry, wherein the one or more contacts are: designed geometrically using a set of rules; arranged with respect to the membrane based on the set of rules or a second set of rules, or both. In some embodiments, the ultrasonic transducer element is a micromachined ultrasonic transducer (MUT) element. In some embodiments, the ultrasonic transducer element is a piezoelectric micromachined ultrasonic transducer (pMUT) element. In some embodiments, the ultrasonic transducer system further comprises: a second ultrasonic transducer element comprising a second substrate and a second membrane; a second electrical circuitry; and one or more additional contacts connected to the second ultrasonic transducer element and the second electrical circuitry, wherein the one or more additional contacts optionally designed geometrically using the set of rules, and wherein the one or more additional contacts are arranged with respect to the second membrane based on the set of rules or the second set of rules. In some embodiments, the first and the second ultrasonic transducer elements form an array with a plurality of additional ultrasonic transducer elements. In some embodiments, the array is two-dimensional. In some embodiments, the array is 32 by 32, 32 by 64, 32 by 194, 12 by 128, 24 by 128, 32 by 128, 64 by 128, 64 by 32, or 64 by 194. In some embodiments, the electric circuitry is an application specific integrated circuit (ASIC). In some embodiments, the one or more contacts comprise at least one contact that is not hybrid contact. In some embodiments, the one or more contacts are electrical contacts only or mechanical contacts only. In some embodiments, the one or more contacts are hybrid contacts. In some embodiments, the one or more contacts comprise at least one electrical contact and one mechanical contact. In some embodiments, the one or more contacts comprise at least one contact that is both electrical and mechanical. In some embodiments, the set of rules comprises one or more of: a range of diameter, a range of height, a range of aspect ratio, and one or more shapes of the one or more contacts. In some embodiments, the range of diameter is about 5 μm to about 100 μm. In some embodiments, the range of height is about 0 μm to about 300 μm. In some embodiments, the aspect ratio of height to effective diameter is less than about 60:1. In some embodiments, the one or more shapes are from: a cylinder, an annular shape, a cubic shape, a cuboid shape, and an elongated shape. In some embodiments, the second set of rules comprises one or more of: a range of spacing of the one or more contacts to the membrane, a minimum number of electrical contacts within the ultrasonic transducer element, a maximum number of electrical contacts within the ultrasonic transducer element, a minimum number of mechanical contacts within the ultrasonic transducer element, a maximum number of mechanical contacts within the ultrasonic transducer element, a minimum number of hybrid contacts within the ultrasonic transducer element, a maximum number of hybrid contacts within the ultrasonic transducer element. In some embodiments, the range of spacing is no less than about 5 μm. The ultrasonic transducer system can have a minimum number of electrical contacts of 2. In some embodiments, the maximum number of electrical contacts is 4. In some embodiments, the minimum number of mechanical contacts is 2. In some embodiments, the minimum number of mechanical contacts is a single contact. In some embodiments, the maximum number of mechanical contacts is 10. In some embodiments, the second set of rules comprises: arranging the one or more contacts to be symmetrical about an axis of the membrane; and arranging the one or more contacts to surround the membrane, or their combination.

In another aspect, disclosed herein are methods of improving performance of an ultrasonic transducer system using hybrid contacts, comprising: obtaining an ultrasonic transducer system, the ultrasonic transducer system comprising: an ultrasonic transducer element comprising a substrate and a membrane; and an electrical circuitry connected to the ultrasonic transducer element; obtaining one or more contacts, the one or more contacts optionally designed geometrically using a set of rules; adding the one or more contacts to the ultrasonic transducer element, comprising: arranging the one or more contacts with respect to the membrane based on the set of rules or a second set of rules; and connecting the one or more contacts to the ultrasonic transducer element and the electrical circuitry.

In yet another aspect, disclosed herein are methods of improving performance of an ultrasonic transducer system using hybrid contacts, comprising: obtaining an ultrasonic transducer system, the ultrasonic transducer system comprising: an ultrasonic transducer element comprising a substrate and a membrane; and an electrical circuitry connected to the ultrasonic transducer element; obtaining one or more contacts, the one or more contacts optionally designed geometrically using a set of rules; adding the one or more contacts to the ultrasonic transducer system, comprising: arranging the one or more contacts with respect to the membrane based on the set of rules or a second set of rules; and connecting the one or more contacts to the ultrasonic transducer element and the electrical circuitry. In some embodiments, the ultrasonic transducer element is a micromachined ultrasonic transducer (MUT) element. In some embodiments, the ultrasonic transducer element is a piezoelectric micromachined ultrasonic transducer (pMUT) element. In some embodiments, the ultrasonic transducer system further comprise: a second ultrasonic transducer element comprising a second substrate and a second membrane; a second electrical circuitry; and one or more additional contacts connected to the second ultrasonic transducer element and the second electrical circuitry, wherein the one or more additional contacts optionally designed geometrically using the set of rules, and wherein the one or more additional contacts are arranged with respect to the second membrane based on the set of rules or the second set of rules. In some embodiments, the first and the second ultrasonic transducer elements form an array with a plurality of additional ultrasonic transducer elements. In some embodiments, the array is two-dimensional. In some embodiments, the array is 32 by 32, 32 by 64, 32 by 194, 12 by 128, 24 by 128, 32 by 128, 64 by 128, 64 by 32, or 64 by 194. In some embodiments, the electric circuitry is ASIC. In some embodiments, the one or more contacts comprise at least one contact that is not a hybrid contact. The method can be performed with one or more contacts being electrical contacts only or mechanical contacts only. In some embodiments, the one or more contacts are hybrid contacts. In some embodiments, the one or more contacts comprise at least one electrical contact and one mechanical contact. In some embodiments, the one or more contacts comprise at least one contact that is both electrical and mechanical. In some embodiments, the set of rules comprises one or more of: a range of diameter, a range of height, a range of aspect ratio, and a shape of the one or more contacts. In some embodiments, the range of diameter is about 5 μm to about 100 μm. In some embodiments, the range of height is about 0 μm to about 300 μm. In some embodiments, the aspect ratio is less than about 60:1. In some embodiments, the shape is one or more selected from: a cylinder, an annular shape, and an elongated shape. In some embodiments, the second set of rules comprises one or more of: a range of spacing of the one or more contacts to the membrane, a minimum number of electrical contacts within the ultrasonic transducer element, a maximum number of electrical contacts within the ultrasonic transducer element, a minimum number of mechanical contacts within the ultrasonic transducer element, a maximum number of mechanical contacts within the ultrasonic transducer element, a minimum number of hybrid contacts within the ultrasonic transducer element, a maximum number of hybrid contacts within the ultrasonic transducer element. In some embodiments, the range of spacing is no less than about 5 μm. In some embodiments, the minimum number of electrical contacts is 2. In some embodiments, the maximum number of electrical contacts is 4. In some embodiments, the minimum number of mechanical contacts is 2. In some embodiments, the minimum number of mechanical contacts is a single contact. In some embodiments, the maximum number of mechanical contacts is 10. In some embodiments, the second set of rules comprises: arranging the one or more contacts to be symmetrical about an axis of the membrane; arranging the one or more contacts to surround the membrane, or their combination.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. A better understanding of the features and advantages of the present subject matter will be obtained by reference to the following detailed description that sets forth illustrative embodiments and the accompanying drawings of which:

FIGS. 1A-1B show a cross-section view and layout view, respectively, of an exemplary embodiment of an integrated MUT and ASIC system using electrical contacts with asymmetry in the electrical contacts (102) (the ASIC die (104) is removed from this figure for clarity purposes);

FIG. 2 shows a layout view of an exemplary embodiment of an integrated MUT and ASIC system using hybrid contacts with added contacts for symmetrical boundary conditions;

FIG. 3 shows exemplary dynamic response of the central MUT membrane in the MUT array of FIG. 1B with asymmetric contacts and the dynamic response of the central MUT membrane in the MUT array of FIG. 2 with symmetric hybrid contacts;

FIG. 4 show exemplary geometrical parameters of a cylindrical electrical and/or mechanical contact of an integrated MUT and ASIC system herein;

FIG. 5 shows exemplary performance of the integrated MUT and ASIC system with hybrid contact arrangement as shown in FIG. 2 with contacts of a 60 μm diameter and three different heights: 6 μm, 16 μm, and 40 μm;

FIG. 6 shows exemplary performance of the integrated MUT and ASIC system with hybrid contact arrangement as shown in FIG. 2 with contacts of a 50 μm diameter (left) and 40 μm diameter (right), at three different heights: 6 μm, 16 μm, and 40 μm;

FIG. 7 shows a layout view of an exemplary embodiment of an integrated MUT and ASIC system using hybrid contacts in FIG. 1B with added hybrid contacts; and

FIG. 8 shows a layout view of an exemplary embodiment of an integrated MUT and ASIC system using hybrid contacts in FIG. 1B with added hybrid contacts.

### DETAILED DESCRIPTION

In some embodiments, a transducer herein is a device that converts a physical variation in one energy domain into a physical variation in a different domain. A micromachined ultrasonic transducer (MUT), for example, converts electrical variations into mechanical vibrations of a diaphragm. These vibrations of the diaphragm result in pressure waves in any gas, liquid, or solid adjoining the diaphragm. Conversely, pressure waves in the adjoining media may cause mechanical vibration of the diaphragm. The diaphragm vibration may in turn result in electrical variations on the MUT's electrodes, which can be sensed. For a piezoelectric MUT (pMUT), an electrical field across the piezoelectric film will change the strain on the diaphragm which may cause the diaphragm to move and subsequently generate pressure waves. Impinging pressure waves from the media onto the pMUT may, in turn, vibrate the diaphragm and create strain in the piezoelectric film which may produce a change in charge on the electrodes of the pMUT.

In certain embodiments, disclosed herein are electrical transducers in which one of the two energy domains is electrical. In some embodiments, disclosed herein are ultrasonic transducers that are electrical transducers. For example, the pMUT (piezoelectric MUT) is an electrical transducer as the electrical domain is one of the energy domains the pMUT converts between while the other domain being mechanical, e.g., mechanical pressure.

The present disclosure includes methods of changing the dynamic behavior of an electrical transducer. In some embodiments, the methods herein are applicable to electrical transducers, ultrasonic transducers, MUT transducers, pMUT transducers, or any other types of transducers. In some embodiments, the methods herein are applicable to electrical transducers other than pMUT, including but not limited to capacitive, piezo-resistive, thermal, optical, radioactive transducers. A piezo-resistive pressure transducer, for example, converts mechanical pressure variations into changes in electrical resistance variations via the piezo-resistance effect. Because the resistance variations are in the electrical domain, the piezo-resistive pressure transducer qualifies as an electrical transducer.

### Certain Definitions

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

The systems and methods herein include a MUT integrated with an ASIC featuring both electrical contacts to transmit/receive signals to/from the ASIC as well as non-electrical contacts (e.g., mechanical contacts) to ensure dynamic performance and reliability of the MUT. In some embodiments, unlike contact arrangements in conventional chip designs, the contacts can be located close to the mechanically sensitive membrane portion of the MUTs, where the membrane vibrates at high frequency band of about 1 MHz to about 10 MHz. As a result, the contact design (e.g., contact type, location, shape, size, etc) can be very important in order to ensure dynamic performance and reliability of the MUT. In some embodiments, the systems and methods herein includes arranging the contacts in such a way to enhance mechanical performance of a MUT (e.g., surface velocity of membrane, magnitude of pressure output, and ultrasonic frequency bandwidth of the MUTs), rather than simply satisfying electrical connectivity and thermal cycle reliability.

In some embodiments, the contact herein connects the elements to which it is attached. In some embodiments, such connection provided by the contact is electrical or non-electrical. In some embodiments, such connection is mechanical. In some embodiments, such connection is mechanical only. In some embodiments, a contact herein is mechanical, although based off its location, the contact may or may not affect the transducer's mechanical operation at a same level. In some embodiments, the contact herein is hybrid, e.g., both electrical and non-electrical. In some embodiments, such hybrid contact herein enables mechanical and electrical contact. In some embodiments, the mechanical contact can be used to carry electrical signals as the electrical contacts, thus making it a hybrid contact. In some embodiments, the hybrid contact may be configured to provide more than one type of connections either simultaneously, or at different time points. For example, a contact may be configured to provide mechanical and electrical connection simultaneously while another contact may be configured to provide electrical connecting but not mechanical connection when a predetermined threshold condition has been met (e.g., a location threshold).

In some embodiments, a hybrid contact array includes more than one type of contacts arranged in one, two, or three dimensions. In some embodiments, a hybrid contact array includes one or more hybrid contacts arranged in one, two, or three dimensions. In some embodiments, the contact herein is a hybrid contact providing any two different type of connections (e.g., electrical and mechanical). In some embodiments, the contact herein is an electrical only (e.g., with no or minimal mechanical effect to the transducer's mechanical operations) and/or mechanical only contact.

In conventional integrated system of MUT and a circuit, e.g., ASIC, the electrical contacts are generally of simple shapes, typically an approximate cylinder shape with a set diameter and height. In conventional integrated system of MUT and ASIC, the position of electrical contacts on the die is typically dictated by the electrical routing of the MUT and the ASIC. Disadvantages can exist in conventional integrated systems as the electrical contacts are often designed (e.g., size, shape, and position, etc.) to achieve thermal cycle reliability with no consideration for MUT performance.

In some embodiments, the MUT here is a MUT array of MUT transducers (interchangeable herein as transducer elements), each MUT transducer having a substrate, a diaphragm (interchangeable here as “membrane”), and/or a piezoelectric element. In some embodiments, the array is in two dimensions. In a MUT array, each MUT transducer acts as a pixel. In some embodiments, the array size may be variable and customized for various applications. Non-limiting exemplary array sizes are: 32 by 32, 32 by 64, 32 by 194, 12 by 128, 24 by 128, 32 by 128, 64 by 128, 64 by 32, or 64 by 194 (columns by rows, or rows by columns).

In some embodiments, the size of each pixel herein is variable and can be customized for various applications. In some embodiments, each pixel herein includes a width (x-axis) and/or height (z axis) that is in the range of about 10 μm to about 1000 μm or 10 μm to 1000 μm. In some embodiments, each pixel herein includes a width (x-axis) that is in the range of about 20 μm to about 600 μm, about 30 μm to about 500 μm, about 40 to about 400 μm, about 50 to about 300 μm, or about 50 μm to about 250 μm. In some embodiments, each pixel herein includes a height that is in the range of about 10 μm to about 1000 μm, about 20 μm to about 950 μm, about 30 to about 900 μm, about 40 to about 850 μm, or about 50 μm to about 800 μm. In some embodiments, the pixel may be asymmetric or symmetric about x, y, z and/or any other axis in the 3D space. In some embodiments, a pixel is taller in the elevation direction, and narrower in the azimuth direction.

In some embodiments, an electrical contact, a mechanical contact, and/or a hybrid contact herein are in close proximity to one or more membranes. In some embodiments, the maximal or minimal distance from an electrical contact 102 or a hybrid contact 105/106 to a membrane 101 is greater than 0 μm, 1μ, 2 μm, 3 μm, 4μ, 5 μ m, 6μ, 7 μm, 8 μm, 9 μm, or 10 μm. In some embodiments, the maximal or minimal distance from an electrical contact or a hybrid contact to a membrane is less than 200 μm, 180 μm, 160 μm, 140 μm, 120 μm, 100 μm, 90 μm, 80 μm, 70 μm, 60 μm, 50 μm, 40 μm, 30 μm, 20 μm, 10 μm or even less, including increments therein. In some embodiments, the maximal or minimal distance from an electrical contact or a hybrid contact to a membrane is in the range of about 10 μm to about 100 μm or 10 μm to 100 μm. In some embodiments, the maximal or minimal distance from an electrical contact or a hybrid contact to a membrane is in the range of about 5 μm to about 150 μm or 5 μm to 150 μm.

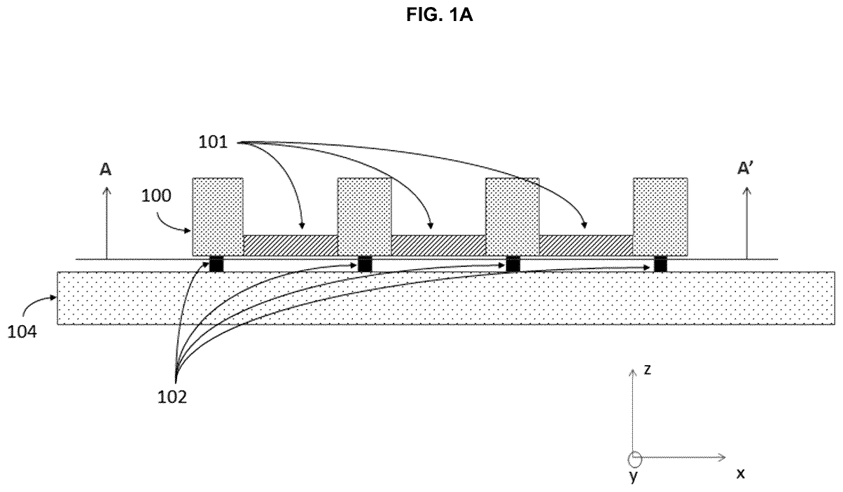

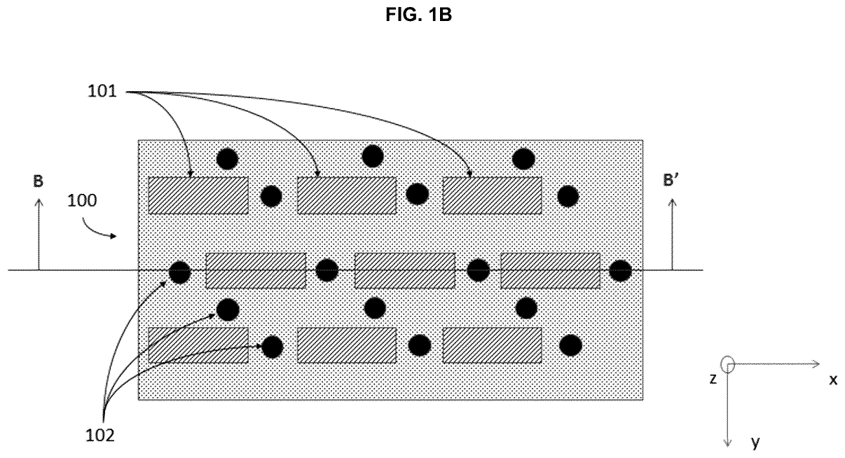

Referring to FIGS. 1A-1B, in a particular embodiment, the integrated system of MUT and ASIC includes a MUT die 100 that is attached or connected to an ASIC die 104 via a plurality of contacts 102. In some embodiments, the ASIC die 104 herein may be other circuit element such as printed circuit board (PCB). The cross-section (at B-B′ in FIG. 1B) and layout views (at A-A′ in FIG. 1A) of the integrated system are shown in FIG. 1A and 1B, respectively. In this embodiment, the MUT die includes an array of transducers which includes an array of membranes 101. The arrangement (e.g., number of membranes per row or column, gaps between adjacent membranes, etc) of such membrane array may be variable, and FIG. 1B shows a non-limiting exemplary arrangement of the array. In this embodiment, the contacts are electrical contacts. In some embodiments, the contacts 102 may be configured to provide additional contacts, such as mechanical contacts. In this embodiment, one or more of the electrical contacts 102 are in close proximity (e.g., with a maximal distance in the range of about 10 μm to about 100 μm) to the mechanically sensitive MUT membranes 101. In some embodiments, one or more electrical contacts 102 is configured to form a critical interface between the MUT and ASIC. In some embodiments, one or more electrical contacts 102, and/or mechanical contacts is configured to act as important boundary condition for the MUT dynamics or performance.

### Arranging Contacts and/or Adding Additional Contacts

In some embodiments, additional contacts are added to the conventional contact configuration, for example, shown in FIGS. 1A-1B. In some embodiments, each of the added contacts can be an electrical contact, a mechanical contact, or a hybrid contact. In some embodiments, the location of the existing and/or additional contacts (e.g., mechanical contacts) is designed to enhance the thermal stability, structural rigidity and/or dynamic performance of the integrated MUT and ASIC system.

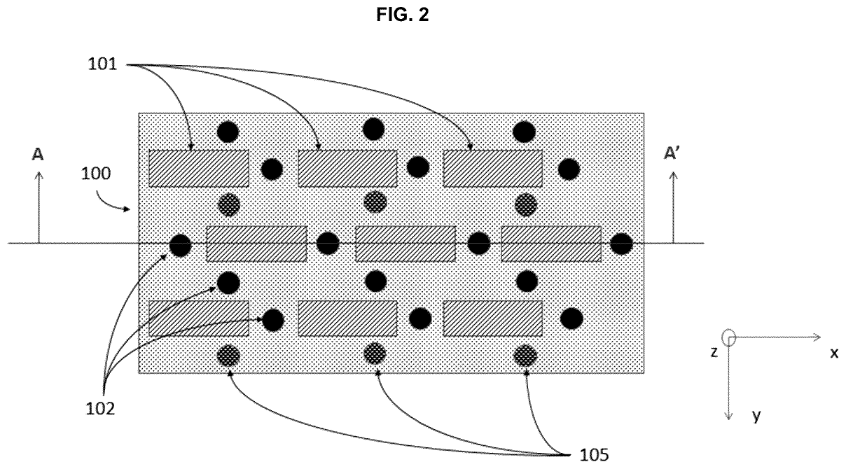

Referring to FIG. 2, in a particular embodiment, additional contacts are added to the existing asymmetric configuration of contacts shown in FIG. 1B. In this embodiment, the mechanical contacts 105 are added to the conventional electrical contact 102 arrangement and the integrated MUT and ASIC system includes a symmetrical layout of contacts, forming a symmetric array of hybrid contacts, in the x-y plane, optionally about the x axis, y axis, or any other axes within the x-y plane. In some embodiments, a symmetric array includes symmetrical arrangement of identical geometrical shapes of contacts, but the contacts may be of different types, e.g., as shown in FIG. 2. In some embodiments, a symmetric array includes symmetrical arrangement of contacts.

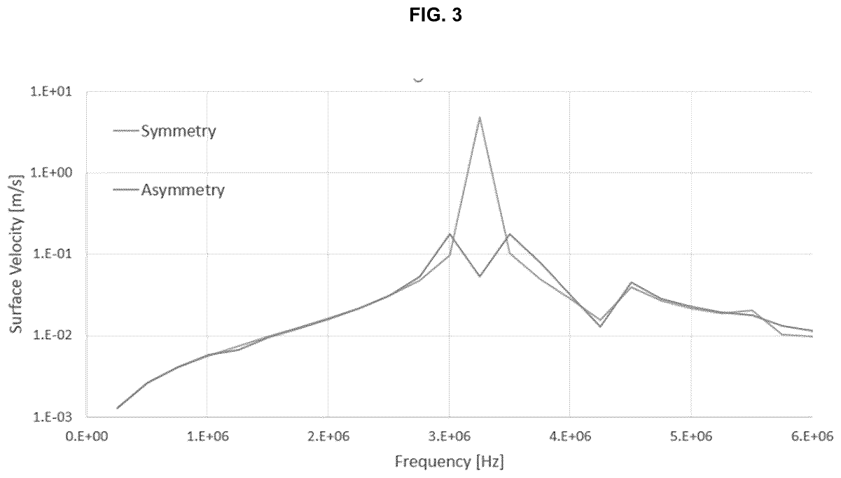

FIG. 3 shows the surface velocity versus frequency of the asymmetric contact arrangement of FIG. 1B in comparison to the symmetric hybrid contact arrangement of FIG. 2. As can be seen in FIG. 3, the symmetrical contact arrangement in FIG. 2 enables a much higher maximal surface velocity than that of the asymmetrical contact arrangement in FIG. 1B. In some embodiments, an advantage of a MUT is that it is configurable to provide a maximum surface velocity of the membrane higher than other types of ultrasound transducers. In some embodiments, the maximum surface velocity of the membrane is directly related to the maximum pressure output achievable with the MUT. Thus, it is advantageous and highly desirable to provide a higher maximum surface velocity with MUT. Referring to FIG. 3, the symmetric hybrid contact arrangement/array in FIG. 2 can achieve more than an order of magnitude higher maximum surface velocity of the membrane. In the same embodiment shown in FIG. 2, the mechanical contacts can offer additional mechanical support that enhances MUT dynamics such as surface velocity of the membrane. For example, the mechanical contact(s) can shift the primary frequency up or down, move harmonic frequencies relative to the primary frequency, thereby affecting the perceived bandwidth of the device. As another example, the contact(s) may increase or decrease the mechanical damping, thus directly affecting the bandwidth of the transducer for its primary and harmonic mode shapes.

In some embodiments, the maximum surface velocity is variable and can dependent on specific applications. In some embodiments, the maximum surface velocity is in the range of about 0.01 m/second to about 100 m/second. In some embodiments, the maximum surface velocity is in the range of about 0.1 m/second to about 10 m/second. In some embodiments, the maximum surface velocity is in the range of about 2 mm/second to about 100 m/second. In some embodiments, the maximum surface velocity is in the range of about 5 mm/second to about 80 m/second. In some embodiments, the maximum surface velocity is in the range of about 5 mm/second to about 60 m/second. In some embodiments, the maximum surface velocity is in the range of about 6 mm/second to about 50 m/second. In some embodiments, the maximum surface velocity is in the range of about 6 mm/second to about 40 m/second. In some embodiments, the maximum surface velocity is in the range of about 6 mm/second to about 30 m/second. In some embodiments, the maximum surface velocity is in the range of about 8 mm/second to about 30 m/second. In some embodiments, the maximum surface velocity is in the range of about 8 mm/second to about 20 m/second. In some embodiments, the maximum surface velocity is in the range of about 8 mm/second to about 15 m/second. In some embodiments, the maximum surface velocity is in the range of about 10 mm/second to about 10 m/second.

In some embodiments, the integrated MUT and ASIC systems herein include a different symmetric hybrid contact array from the one shown in FIG. 2. Such different symmetric hybrid contact array is configured to improve MUT dynamics over conventional contact arrays. In some embodiments, the symmetric hybrid contact array is symmetrical about x-axis, y axis or any other axes within the x-y plane of the MUT die 100. In some embodiments, the symmetric hybrid contact array herein includes contacts that are symmetrically positioned about the x-axis, y-axis or any other axes within the x-y plane of individual MUT membrane 101. In some embodiments, symmetry herein includes: size, shape, type, position, or their combinations of the contact(s). For example, two different contacts (e.g., one hybrid, one electrical) positioned symmetrically about a MUT membrane may be considered a symmetrical arrangement of such two contacts.

In some embodiments, the systems and methods herein includes a MUT array with an arbitrary number of membranes. In some embodiments, the total number of membranes in the MUT array is in the range of 1 to 15,000. In some embodiments, the number of membranes in the MUT array is in the range of 250 to 4,200.

In some embodiments, the systems and methods herein includes a MUT array with an arbitrary number of contacts or hybrid contacts. In some embodiments, the number of contacts or hybrid contacts in the MUT array is in the range of 2 to 120,000. In some embodiments, the number of contacts or hybrid contacts in the MUT array is in the range of 250 to 8,500.

### Designing the Hybrid Contacts

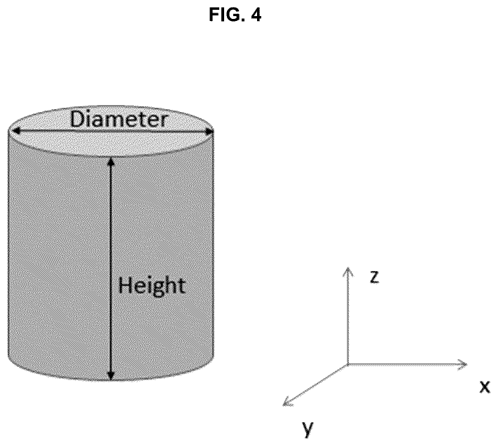

In some embodiments, arrangement of the hybrid contacts is not the sole parameter of the systems and methods herein. The contacts themselves can be designed to further optimize MUT performance. Typically, the contact materials can be set by the integration technology, and so are fixed. As noted previously, in the cases where contacts are typically cylindrical, the contact height (along z axis) and diameter (x-y plane) can be parameters for optimization, as shown in FIG. 4.

In some embodiments, MUT performance can be improved or optimized for a given MUT design and integration scheme using a first set of rules, a second set of rules, or their combinations. The first set of rules, for each contact, can include: a range of diameter, a range of height, range of cross section area, a range of aspect ratio, a shape of the one or more contacts, and a cross-section shape of the one or more contacts. The second set of rules, which may provide for each contact one or more of: a range of spacing of the one or more contacts to the membrane, a minimum number of electrical contacts within the ultrasonic transducer element, a maximum number of electrical contacts within the ultrasonic transducer element, a minimum number of mechanical contacts within the ultrasonic transducer element, a maximum number of mechanical contacts within the ultrasonic transducer element, a minimum number of hybrid contacts within the ultrasonic transducer element, a maximum number of hybrid contacts within the ultrasonic transducer element, a maximum contact area with the ultrasonic transducer element, a minimum contact area with the ultrasonic transducer element. Thus, using one or more such rules may help optimize the MUT performance. For example, a contact shape may be given as cylindrical, and the integration scheme may be provided to be via hybrid contact. The other rules of the first and/or the second set of rules can be selected, either manually, empirically, automatically, or using machine learning algorithms to determine features of the contacts thus optimize MUT performance.

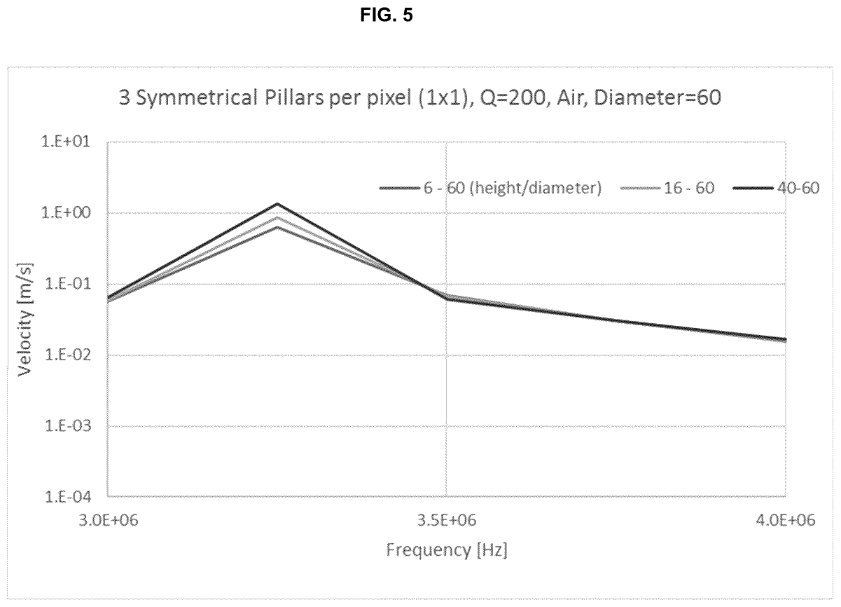

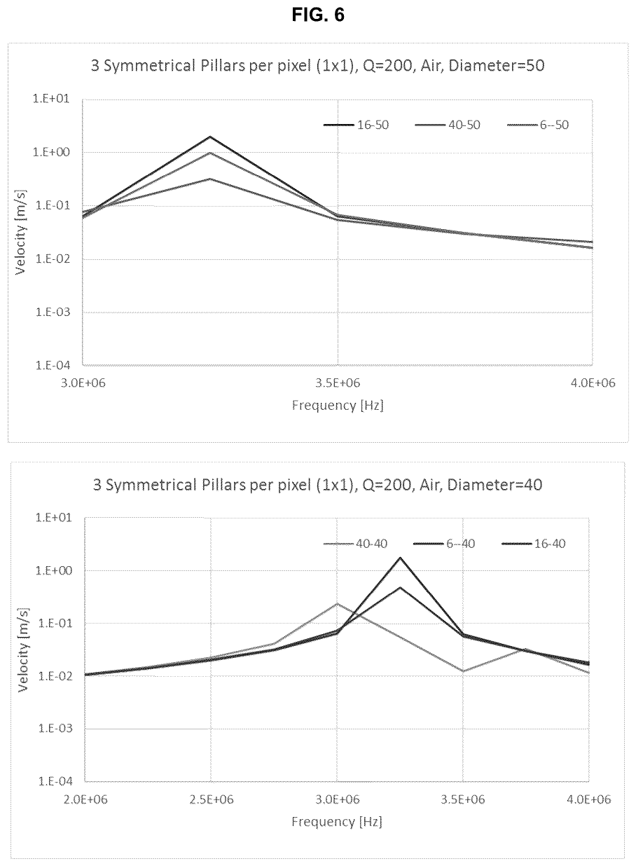

FIG. 5 shows the dynamic performance of the MUT membrane 101 of the hybrid contact configuration illustrated in FIG. 2. In this particular embodiment, the contact with the tallest height of three different heights, i.e., 40 μm, has the best performance compared to 6 μm and 16 μm high contacts, for an identical contact diameter of 60 μm. If the contact diameter is adjusted to 50 μm, the best contact height is 16 μm as illustrated in FIG. 6 (left). For a contact diameter of 40 μm, the best contact height is 6 μm as in FIG. 6 (right).

In some embodiments, the contacts herein can be of shapes other than cylindrical shapes to achieve optimized MUT performance. Non-limiting examples of three dimensional contact shape includes a part or entirety of: a sphere, pyramid, baseball, spindle shape, cube, cuboid, tetrahedron, cone, hexagonal prism, triangular prism, and donut shape. Non-limiting examples of contact shapes along the x-y plane include a part or entirety of: a circle, ring, fan, oval, triangle, square, rectangular, trapezoid, rhomboid, and polygon.

In some embodiments, the contact herein includes a height (along z axis) that can be variable and customized for different applications. In some embodiments, the contact herein includes a height (along z axis) in the range of about 0 μm to 300 μm. In some embodiments, the contact herein includes a height in the range of about 0 μm to 250 μm. In some embodiments, the contact herein includes a height in the range of about 0 μm to 200 μm. In some embodiments, the contact herein includes a height in the range of about 0 μm to 100 μm. In some embodiments, the contact herein includes a height in the range of about 1 μm to 100 μm. In some embodiments, the contact herein includes a height in the range of about 1 μm to 80 μm. In some embodiments, the contact herein includes a height in the range of about 2 μm to 80 μm. In some embodiments, the contact herein includes a height in the range of about 2 μm to 60 μm. In some embodiments, the contact herein includes a height in the range of about 3 μm to 60 μm. In some embodiments, the contact herein includes a height in the range of about 3 μm to 50 μm.

In some embodiments, the contact herein includes a diameter (in x-y plane) that can be variable and customized for different applications. In some embodiments, the contact herein includes a diameter (in x-y plane) in the range of about 0 μm to 300 μm. In some embodiments, the contact herein includes a diameter in the range of about 0 μm to 250 μm. In some embodiments, the contact herein includes a diameter in the range of about 4 μm to 120 μm. In some embodiments, the contact herein includes a diameter in the range of about 5 μm to 100 μm. In some embodiments, the contact herein includes a diameter in the range of about 10 μm to 90 μm. In some embodiments, the contact herein includes a diameter in the range of about 15 μm to 80 μm. In some embodiments, the contact herein includes a diameter in the range of about 20 μm to 80 μm. In some embodiments, the contact herein includes a diameter in the range of about 25 μm to 75 μm. In some embodiments, the contact herein includes a diameter in the range of about 25 μm to 70 μm. In some embodiments, the contact herein includes a diameter in the range of about 30 μm to 60 μm.

In some embodiments, the contact herein includes an aspect ratio (i.e., height: diameter) (that can be variable and customized for different applications. Non-limiting examples of aspect ratio includes but is not limited to: less than 6:1, less than 5:1, less than 4:1, less than 3:1, or less than 2:1. In some embodiments, the aspect ratio is less than 1:1. In some embodiments, the aspect ratio is less than 0.9:1. In some embodiments, the aspect ratio is less than 0.8:1. In some embodiments, the aspect ratio is less than 0.7:1. In some embodiments, the aspect ratio is less than 0.6:1. In some embodiments, the aspect ratio is less than 0.5:1. In some embodiments, the aspect ratio is less than 0.4:1. In some embodiments, the aspect ratio is less than 0.3:1. In some embodiments, the aspect ratio is less than 1.2:1. In some embodiments, the aspect ratio is less than 0.2:1. In some embodiments, the aspect ratio is less than 0.8:1. In some embodiments, the aspect ratio is less than 1.5:1.

In some embodiments, the contacts herein include a contact area in the x-y plane. In some embodiments, the contact area contacts the transducer element and/or the circuit herein. In some embodiments, the contact area is a cross section perpendicular to the z-axis. In some embodiments, the contact area is equivalent to a circle of diameter ranging from 30 μm to 60 μm.

In some embodiments, the contact area is equivalent to a circle of diameter ranging from 10 μm to 100 μm. In some embodiments, the contact area is equivalent to a circle of diameter ranging from 20 μm to 80 μm. In some embodiments, the contact area is equivalent to a circle of diameter ranging from 30 μm to 50 μm. In some embodiments, the contact area is equivalent to a circle of diameter ranging from 40 μm to 60 μm.

In some embodiments, each pixel herein includes one or more electrical, mechanical, and/or hybrid contacts. In some embodiments, each pixel includes 1 to 5, 1 to 4, 1 to 3, or 1 to 2 electrical contacts. In some embodiments, each pixel includes 1 to 10, 1 to 8, 1 to 6, or 1 to 5, 1 to 4, 1 to 3, or 1 to 2 mechanical contacts. In some embodiments, each pixel includes 1 to 10, 1 to 8, 1 to 6, or 1 to 5, 1 to 4, 1 to 3, or 1 to 2 hybrid contacts.

### Shaping Hybrid Contacts

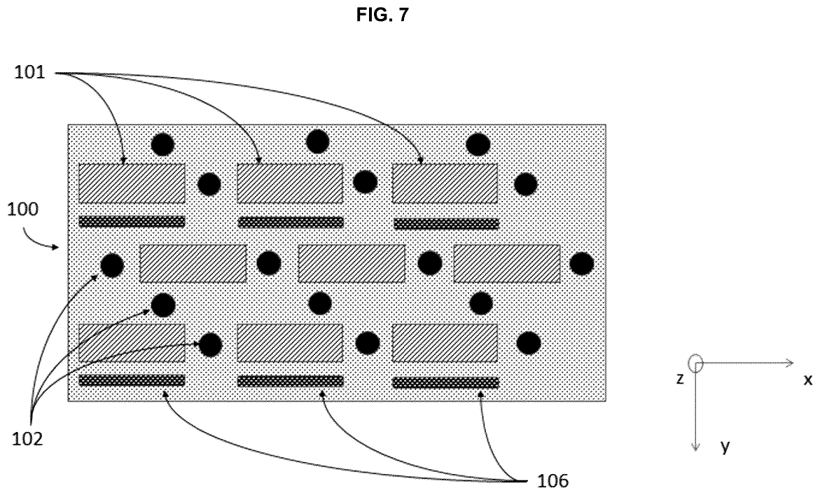

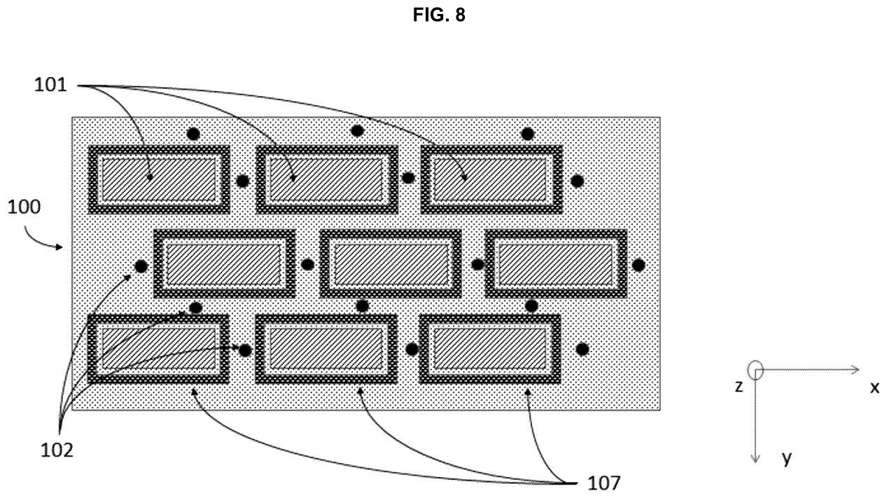

In some embodiments, the hybrid contacts may not be of a uniform layout shape. Instead, in some embodiments, the hybrid contacts may be with a variety of shapes that facilitate improvement of MUT performance. As an example, FIG. 7 illustrates added elongated contacts 106 that are longer (e.g., along x axis) and slender (e.g., along y axis). The contact 106 can be hybrid contact or mechanical contact. In this embodiment, such elongated contacts provide a large fixed boundary condition thus improving the MUT performance. As another example, one or more of the membranes 101 of the MUT array 100 may be enclosed at least partly by the annular hybrid contacts 107 illustrated in FIG. 8. In these embodiments, the annular contacts and the elongated contacts are hybrid. In some embodiments, the annular contacts and the elongated contacts are electrical and/or mechanical.

Although certain embodiments and examples are provided in the foregoing description, the inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein A and/or B encompasses one or more of A or B, and combinations thereof such as A and B. It will be understood that although the terms “first,” “second,” “third,” etc. may be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a,” “an,” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20%, including increments therein, of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. An ultrasonic transducer system comprising a micromachined ultrasonic transducer (MUT) element electrically connected to electrical circuitry by one or more hybrid contacts having uniform cross sections that facilitate rigid structural connections between the MUT and the electrical circuitry to enhance a dynamic performance of the ultrasonic transducer system.

2. The ultrasonic transducer system of claim 1, further comprising an additional MUT element electrically connected to additional electrical circuitry by one or more additional contacts, wherein the one or more additional contacts comprise one or more of electrical contacts, mechanical contacts, or additional hybrid contacts, the additional hybrid contacts also having the uniform cross sections.

3. The ultrasonic transducer system of claim 2, further comprising a plurality of MUT elements, wherein the MUT element, the additional MUT element, and the plurality of MUT elements form a two-dimensional array.

4. The ultrasonic transducer system of claim 1, wherein a space between each hybrid contact of the one or more hybrid contacts is at least about 5 μm.

5. The ultrasonic transducer system of claim 1, wherein the one or more hybrid contacts are positioned symmetrically relative to the MUT element.

6. The ultrasonic transducer system of claim 1, further comprising one or more electrical contacts that couple the MUT element to the electrical circuitry.

7. The ultrasonic transducer system of claim 6, wherein the one or more electrical contacts comprises between about two and about four electrical contacts.

8. The ultrasonic transducer system of claim 1, further comprising one or more mechanical contacts that couple the MUT element to the electrical circuitry.

9. The ultrasonic transducer system of claim 8, wherein the one or more mechanical contacts comprises between about two and about ten mechanical contacts.

10. The ultrasonic transducer system of claim 1, wherein the one or more hybrid contacts are configured to provide electrical connection and mechanical connection simultaneously.

11. The ultrasonic transducer system of claim 1, wherein the one or more hybrid contacts are configured to provide only electrical connection before a predetermined threshold condition has been met.

12. The ultrasonic transducer system of claim 1, wherein a ratio of a height of the one or more hybrid contacts to a diameter of the one or more hybrid contacts is between about 1:2 and about 2:1.

13. The ultrasonic transducer system of claim 1, wherein a ratio of a height of the one or more hybrid contacts to a diameter of the one or more hybrid contacts is about 1:1.

14. The ultrasonic transducer system of claim 1, wherein a height of the one or more hybrid contacts is about 50 μm and a diameter of the one or more hybrid contacts is about 50 μm.

15. The ultrasonic transducer system of claim 1, wherein the uniform cross section is annular.

16. A method of improving performance of an ultrasonic transducer system by mechanically and electrically coupling micromachined ultrasonic transducer (MUT) elements to electrical circuitry via one or more hybrid contacts, wherein each hybrid contact of the one or more hybrid contacts comprises a uniform cross section that stabilizes the mechanical coupling between the MUT elements and the electrical circuitry.

17. The method of claim 16, wherein mechanically and electrically coupling the MUT elements to the electrical circuitry via one or more hybrid contacts comprises mechanically and electrically coupling a first MUT element to a first electrical circuitry via a first set of hybrid contacts and mechanically and electrically coupling a second MUT element to a second electrical circuitry via a second set of hybrid contacts.

18. The method of claim 16, further comprising, before mechanically and electrically coupling the MUT elements to the electrical circuitry via one or more hybrid contacts, arranging the MUT elements in a two-dimensional array.

19. The method of claim 16, further comprising arranging the one or more hybrid contacts symmetrically around each MUT element.

20. The method of claim 16, further comprising coupling the MUT elements to the electrical circuitry via mechanical contacts or electrical contacts.
